(19) United States
(12) Patent Application Publication
(10) Pub. No.: US 2006/0234348 A1
(43) Pub. Date: Oct. 19, 2006

(54) Method of production of RNA polymerase protein

(76) Inventors: Jozef J. Bujarski, DeKalb, IL (US); Rafal Wierzchoslawski, Frederick, MD (US)

Correspondence Address: Kenneth I. Kohn;Kohn & Associates, PLLC, Suite 410, 30500 Northwestern Hwy., Farmington Hills, MI, 48334, US

(21) Appl. No.: 11/403,570

(22) Filed: Apr. 13, 2006

Related U.S. Application Data
(60) Provisional application No. 60/670,780, filed on Apr. 13, 2005.

(51) Int. Cl. C12P 21/06; C12N 9/22; C07H 21/04; C12N 15/74; C12N 1/21

(52) U.S. Cl. 435/69.1; 435/199; 435/252.3; 435/471; 536/23.2

(57) ABSTRACT

A method of forming a stable RNA polymerase enzyme by expressing protein 2a in cells, pelleting the cells expressing protein 2a, macerating the cells expressing protein 2a to obtain a cell lysate, and filtering the cell lysate through an affinity resin. A stable RNA polymerase enzyme for copying RNA in vitro for use is disclosed.

### CROSS REFERENCE TO RELATED APPLICATIONS

This application claims the benefit of priority under 35 U.S.C. § 119(e) of U.S. Provisional Patent Application Ser. No. 60/670,780, filed Apr. 13, 2005, which is incorporated herein by reference.

### GRANT INFORMATION

Research in this application was supported in part by a grant from the National Science Foundation: MCB-0317039. The Government has certain rights in the invention.

### BACKGROUND OF THE INVENTION

1. Technical Field

The present invention relates to the production of protein 2a. More specifically, the present invention relates to the production of protein 2a that can copy, in vitro, any RNA.

2. Description of the Related Art

The quality of an RNA preparation greatly affects the results obtained from RNA analysis techniques used in molecular biology, such as Northern blotting, ribonuclease protection assays and RT-PCR (Reverse Transcriptase-Polymerase Chain Reaction). Degraded RNA sample will produce a lower signal than an equivalent intact RNA.

RNA is much more susceptible to degradation than DNA (Sambrook et al., 1989). RNA is readily hydrolyzed when exposed to conditions of high pH, metal cations, high temperatures, and ribonucleases. A major cause of RNA degradation is ribonuclease contamination, which must be avoided in virtually all RNA-related procedures, including RNA isolation, mRNA purification, RNA storage, Northern blotting, nuclease protection assays, RT-PCR, in vitro transcription and/or translation, and RNA diagnostics. In addition to the endogenous ribonucleases from cells and tissues, finger grease and bacteria and/or fungi in airborne dust particles are common sources of ribonuclease. To minimize ribonuclease contamination, appropriate precautions must be followed when handling RNA (Blumberg, 1987; Wu, 1997).

Ribonucleases are difficult to inactivate. For example, bovine pancreatic ribonuclease A (RNase A) displays no activity at 90° C. However, the activity is restored upon quick cooling to 25° C. This process is known as reversible thermal denaturation. An incubation of RNase A at 90° C. for the prolonged periods of time, results in decreased activity of fractions recovered at 25° C., due to irreversible thermoinactivation. At 90° C., it takes several hours to inactivate RNase A (Zale and Klibanov, 1986). The loss of activity is attributed to disulfide interchange (Zale and Klibanov, 1986). Several ribonucleases can even withstand autoclaving (121° C., 15 psi, 15 minutes) to some degree. Furthermore, Spackman et al. (1960) observed that RNase A was stable at elevated temperatures, extreme pH values, and in presence of protein denaturants, and urea. These observations emphasized the difficulty researchers have had inactivating ribonucleases. For the above reason, a variety of methods, other than heating, have been developed to inhibit or inactivate ribonucleases.

One method of destroying RNases involves the treatment of molecular biology reagents, glassware or electrophoresis apparatus with a 0.1% solution of diethyl pyrocarbonate (DEPC), followed by incubation at 37° C. for several hours and then autoclaving for 15-20 minutes to destroy the DEPC (Wolf et al., 1970). DEPC reacts with the epsilon-amino groups of lysine and the carboxylic groups of aspartate and glutamate both intra- and intermolecularly (Wolf et al., 1970). The chemical reaction forms polymers of the ribonuclease. However, there are several disadvantages of using DEPC: (i) DEPC is a possible carcinogen, posing hazard to humans; (ii) some commonly used molecular biology reagents such as Tris react with and inactivate DEPC; (iii) treatment of samples with DEPC is time-consuming; (iv) DEPC reacts with the adenine residues of RNA, rendering it inactive in the in vitro translation reactions (Blumberg, 1987) and (v) the remaining DEPC residues not destroyed by autoclaving, may inhibit subsequent enzymatic reactions.

RNA may be stored in a number of ways. For short-term storage, RNase-free H₂O (with 0.1 mM EDTA) or TE buffer (10 mM Tris, 1 mM EDTA) may be used. RNA is generally stable at −80° C. for up to a year. Magnesium and other metals catalyzing non-specific cleavages in RNA should be eliminated by the addition of the chelating agent such as EDTA if RNA is to be stored and retrieved intact. It is important to use EDTA-containing solution known to be RNase-free for this purpose. Older EDTA solutions may have microbial growth that could contaminate the RNA sample with nucleases. It has been suggested that RNA solubilized in formamide may be stored at −20° C. without degradation for at least one year (Chomczynski, 1992).

More specifically, for long-term storage, RNA samples can be stored at −20° C. as ethanol precipitates. Accessing these samples on a routine basis can be a nuisance, however, since the precipitates must be pelleted and dissolved in an aqueous buffer before pipetting, if accurate quantitation is important. Alternatively, the sample can be pipetted directly out of an ethanol precipitate that has been vortexed to create an even suspension. While this method is suitable for qualitative work, it is too imprecise for use in quantitative experiments. RNA does not disperse uniformly in ethanol, probably because it forms aggregates; non-uniform suspension, in turn, leads to inconsistency in the amount of RNA removed when equal volumes are pipetted.

For long-term storage, RNA samples are best stored as salt/ethanol slurry. To do this, the RNA is taken through all the steps of a regular precipitation with salt (e.g., 1/10 volume of 3 M NaOAc, pH 4.8) and ethanol (2 volumes of 100% ethanol) and the mixture is stored at −80° C. without pelleting the RNA out of the solution. The combination of low pH, low temperature and high alcohol content will stabilize the RNA and inhibit all enzymatic activity. Alternatively, the RNA can be long-term stored in formamide (Chomczynski, 1992) or in frozen aliquots at −20° C. or below. If stored in formamide or ethanol, the RNA will need to be pelleted prior to quantitation or other manipulation.

For short-term storage, RNA samples can be resuspended in water or buffer and stored at −80° C. If water is the preferred medium, nuclease-free water must be used. Using a buffer solution that contains a chelating agent is a better way to store RNA. Chelation of divalent cations such as Mg⁺² and Ca⁺² prevents heat-induced strand scission; RNA can be chemically cleaved when heated in the presence of Mg⁺². Ambion provides nuclease-free water and a variety of buffers, including TE, 0.1 mM EDTA and RNA Storage Solution, which has the added benefit of a low pH, for storing RNA.

While RNA can be stored in DEPC-treated water or in TE buffer, it is not protected from degradation if the sample or the storage solution has a minor ribonuclease contamination. It has been suggested that RNA be stored in ethanol or formamide to protect sample from degradation because these environments minimize ribonuclease activity (Chomczynski, 1992). The obvious disadvantage is that the RNA sample cannot be directly utilized for analysis or enzymatic reactions unless the ethanol or formamide is removed.

Guanidinium thiocyanate is commonly used to inhibit RNases during RNA isolation (Chomczynski and Sacchi, 1987; Sambrook et al., 1989). A high concentration of guanidinium thiocyanate combined with P-mercaptoethanol is used to isolate RNA from tissues, even those that are rich in ribonucleases, such as pancreas (Chirgwin et al., 1979). Guanidinium thiocyanate is an effective inhibitor of most enzymes due to its chaotropic nature. However, the RNA stored in this manner must be purified from the guanidinium thiocyanate residues prior to being used in an enzymatic reaction.

Vanadyl-ribonucleoside complexes (VRC) may be used to inhibit RNases during RNA preparation (Berger and Birkenmeier, 1979). The drawback to using VRC, is that VRC strongly inhibits the translation of mRNA in cell-free systems and must be removed from RNA samples by phenol extraction (Sambrook et al., 1989).

Favaloro et al. (1980) employed macaloid, (a form of clay), to absorb RNases. A limitation of this method is that it is difficult to completely remove the clay from RNA samples. Other reagents have been used to inhibit ribonucleases including SDS, EDTA, proteinase K, heparin, hydroxylamine-oxygen-cupric ion, bentonite and ammonium sulfate (Allewell and Sama, 1974; Jocoli and Ronald, 1973; Lin, 1972; Jones, 1976; Mendelsohn and Young, 1978). None of these reagents are strong inhibitors alone, although their inhibitory effect may be improved by using them in combination. Like many of the RNase inhibitors already described, while these chemicals inhibit RNase activity, they may also inhibit other enzymes such as reverse transcriptase and DNase I. Therefore, the RNA must be purified from the inhibitory reagent(s) before it can be subjected to other enzymatic processes.

Two types of proteinaceous RNase inhibitors are commercially available: human placental ribonuclease inhibitor (Blackburn et al., 1977) and PRIME Inhibitor™ (Murphy et al., 1995). RNases of the class A family bind tightly to these protein inhibitors and form noncovalent complexes that are enzymatically inactive. The major disadvantage of these inhibitors is that they have a narrow spectrum of specificity. Also, the inhibitors do not inhibit other than A, classes of RNases. Another disadvantage when using placental ribonuclease inhibitor is that it denatures within hours at 37° C., releasing the bound ribonuclease. Thus, the RNA sample is only protected for a few hours at the most.

Reducing agents are frequently used as adjuvants to RNA isolation solutions, in conjunction with denaturants to reduce the disulfide bonds in RNases that are rendered accessible by the denaturant. Commonly used reducing reagents are beta-mercaptoethanol, dithiothreitol (DTT), dithioerythritol (DTE), glutathione, or amino acid cysteine. Beta-mercaptoethanol is often included in RNA isolation solutions combined with guanidinium thiocyanate to reduce ribonuclease activity and solubilize proteins (Chomcyznski and Sacchi, 1987). DTT is the strongest reducing reagent of the three listed.

DTT has a low redox potential (−0.33 volts at pH 7.0) and is capable of maintaining monothiols effectively in the reduced form and of reducing disulfides quantitatively (Cleland, 1964). DTT acts as a protective agent for free sulfhydryl groups. It is highly water soluble, with little tendency to be oxidized directly by air, and is superior to other thiols used as protective reagents. DTT's reducing activity can be accurately assayed using 5,5′-dithiobis (2-nitrobenzoic acid) or DTNB (Cleland, 1964). The reduction of DTNB mediated by DTT generates a yellow color, which absorbance can be measured at 412 nm using a spectrophotometer. RNase A, RNase 1 and RNase T1, all contain disulfide bonds (Ryle and Anfinesen, 1957; Barnard, 1969) and therefore, are susceptible to reduction.

DTT has been used as an inhibitor of RNase A in the isolation of polyribosomes (Boshes, 1970; Aliaga, 1975). Polyribosome preparations were treated with RNase A (10 μg/ml) in solution A (10 mM MgCl.sub.2: 10 mM Tris [pH 7.6]: 50 mM KCl) in the presence or absence of 4 mM DTT at 4° C. for 20 minutes. The treatment of polyribosomes with RNase A in the absence of DTT generated monoribosomes. Polyribosomes treated with RNase A in the presence of 4 mM DTT reduced the conversion of polyribosomes to monoribosomes, hence, it was concluded that DTT was an RNase inhibitor. The RNase inhibitors require a reducing environment for activity.

Thus, the in vitro storage of single-stranded RNAs (ssRNAs) poses a challenge and by requiring a ribonuclease-free environment (RNase-free water or special buffers). Converting ssRNA into a double-stranded (ds) form significantly protects the RNA against degradation. Presently, the RNA of interest is reverse-transcribed from a mixture of RNAs as a radioactive complementary cDNA strand after initiation with a selective DNA primer. Then, the mixture of RNAs is separated on a gel and the radioactive cDNA product of interest is detected and sized. This protocol allows for elimination of the hybridization step with a highly-radioactive probe. Furthermore, current RNAi techniques often require the use of several sets of single- (antisense) or double-stranded RNA oligonucleotides covering various regions of the silenced mRNA message.

It would therefore be useful to develop a method for improving RNA storage stability.

### SUMMARY OF THE INVENTION

According to the present invention, the method is provided for obtaining a stable RNA polymerase enzyme by expressing protein 2a in cells, pelleting the cells expressing protein 2a, macerating the cells expressing protein 2a to obtain a cell lysate, and filtering the cell lysate through an affinity resin. A stable RNA polymerase enzyme for copying RNA in vitro is also provided.

### DETAILED DESCRIPTION OF THE INVENTION

The invention provides the methodology of production of protein 2a, an active RNA polymerase enzyme, which copies in vitro any RNA. The method of the present invention also provides conditions ensuring enzyme activity and storage stability.

The present invention provides the first RNA-dependent RNA polymerase that has been produced to be stable on a large scale for commercial purposes. The enzyme directly copies any RNA to a complementary strand and can utilize primers so that the initiation site can be custom-addressed. Protein 2a can be used for both RNA activity and storage stability. Such duality depends upon the conditions under which the protein is placed. Under certain conditions the proteins initiates from a specific promoter and under alternative conditions the protein initiates from a complementary primer.

“Brome mosaic virus” as used herein is a plant RNA virus that is not infectious to humans and animals.

“Escherichia coli” as used herein is a bacteria commonly used for protein expression. The E. coli strain used for expression is named ‘Tuner’, and is commercially available.

“Plasmid 2a-JBWA” as used herein is the plasmid construct (based on commercially available expression plasmid PET 15b) that carries the full-length insert of protein 2a open reading frame.

An “affinity column” as used herein is the cobalt-based affinity resin (called TALON resin, commercially available) that interacts specifically with the 6-His epitope tag, enabling the separation of the expressed 2a protein (His-tagged) from the rest of the bacterial lysate components. Any His-tag affinity protocols can be used, including a variety of Ni and Co-based beads. The best performing column was Tallon affinity resin from BD Biosciences. Other similar affinity columns having similar properties to the above affinity column can also be used without departing from the spirit of the present invention.

“Growth medium” as used herein is intended to include any medium capable of growing the desired products. Preferably, a dextrose-enriched TB medium is used as a growing media for bacteria, although other growing media could be used as well.

The present invention can be used for selective copying of RNA of interest from any RNA mixture by using the selective RNA primer complementary to the RNA of interest. This can be used for detection (quantitative or qualitative) of the RNA and as such it provides a convenient alternative to Northern blotting, especially for sizing/cloning of mRNAs in view of their cognate genes. The technique can be useful in assessing up-regulation or down-regulation of the transcription level of particular genes (and thus, gene expression level) upon changes in the environmental conditions, as an alternative to widely used microarraying technologies.

Additionally, the present invention can be used for the down-regulation of transcription by means of RNAi technology. By making the double stranded RNAs with the transgenically expressed 2a protein it is possible to trigger the silencing of a particular gene of interest in transgenic organisms (“selective priming”). The selective priming is achieved with a specific complementary RNA oligonucleotide that can be either transcribed simultaneously with 2a mRNA from the same transgene or from a separate transgene controlled by an inducible promoter. The approach, when applied for the induction of anti-viral resistance, represents alternative or complementary techniques to those already in the use.

The present invention can also be used for the production of desired messenger RNAs in the cytoplasm. The RNA template complementary to the mRNA of interest can be delivered to the cytoplasm using viral particles as a vehicle. The carrier particles are capable of uncoating but are not capable of replication or cell-to-cell spread. The transgenically-expressed 2a protein copies the delivered RNA template into messenger-sense RNA using selective RNA primer. The technique allows circumvention of the potential problems with mRNA editing, splicing, or nuclear export. Moreover, the transient gene expression is achieved only in the selected cells that is, those cells directly infected with the carrier virus. The potential interference by cellular RNAi/PTGS mechanism is minimized by expressing known RNAi suppressors.

Molecular diagnostics of the messenger RNAs produced by the present invention can also be accomplished. By using the selective primers complementary to exons and/or introns of a particular mRNA of interest, the potential defects in RNA splicing/editing could be detected. RNA copying can be accomplished using an RNA primer or by using special signal sequences.

Any method of expression can generate an active 2a RNA polymerase, for example, expression in yeast, expression in plants, and expression in animal/human cells: Further, any method of generating RNA primer can also function as disclosed above.

The cDNA fragment encoding the protein 2a RNA polymerase was ligated into the pET-15b expression vector. The E. coli bacteria were transformed with the resulting plasmid construct, and the transformed bacteria were grown in a medium. The expression of 2a protein was induced by adding a chemical compound (IPTG) and the bacteria were further grown. The bacterial cells were pelleted down, and macerated in a special buffer to obtain a cell lysate. The lysate was filtered through an affinity resin and the resin-bound protein was washed out using an elution buffer. The protein preparation was dialyzed against a storage buffer and stored at low temperature.

Protein 2a can be expressed in cells as an RNA copying agent or, alternatively, an RNA-expressing construct can be introduced into cells for further copying with 2a. For example, 2a can be expressed together with a specific RNA primer, so a selected RNA can be copied into a double-stranded form that thereafter induces specific gene silencing.

By gene therapy as used herein one refers to the transfer of genetic material (e.g. DNA or RNA) of interest into a host to treat or prevent a genetic or acquired disease or condition phenotype. The genetic material of interest encodes a product (e.g. a protein, polypeptide, peptide, functional RNA, or antisense RNA) whose production in vivo is desired. For example, the genetic material of interest can encode a hormone, receptor, enzyme, polypeptide or peptide of therapeutic value. Alternatively, the genetic material of interest encodes a suicide gene. For a review see, in general, “Gene Therapy” (Advances in Pharmacology, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In the in vivo gene therapy, target cells are not removed from the subject but rather the genetic material to be transferred is introduced into the cells of the recipient organism in situ, which is within the recipient/subject. In an alternative embodiment, if the host gene is defective, the gene is repaired in situ. These genetically altered cells have been shown to express the transfected genetic material in situ.

The gene expression vehicle is capable of delivery/transfer of heterologous nucleic acid into a host cell. The expression vehicle can include elements to control targeting, expression and transcription of the nucleic acid in a cell selective manner as it is known in the art. It should be noted that often the 5′UTR and/or 3′UTR of the gene can be replaced by the 5′UTR and/or 3′UTR of the expression vehicle. Therefore as used herein the expression vehicle can, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any non-translated DNA sequence that work contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et at., Gene Targeting, CRC Press, Ann Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature.

Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus-derived vector Adenop53TK. The vector expresses a herpes virus thymidine kinase (TK) gene for either positive or negative selection and an expression cassette for desired recombinant sequences. The vector can be used to infect cells that have an adenovirus receptor that includes most cancers of epithelial origin as well as others. The vector, as well as others that exhibit similar desired functions, can be used to treat a mixed population of cells and can include, for example, an in vitro or ex vivo culture of cells, a tissue, or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the present invention depends on the desired cell type being targeted as is known to those skilled in the art. For example, if breast cancer is to be treated, then a vector specific for such epithelial cells can be used. Likewise, if diseases or pathological conditions of the hematopoietic system are to be treated, then a viral vector that is specific for blood cells and their precursors, preferably for the specific type of hematopoietic cell, can be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles that are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed do not contain a genome and therefore cannot proceed through subsequent rounds of infection. The specific type of vector depends upon the intended application. The actual vectors are also known and readily available to one skilled in the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment. Administration can also be performed by, for example, intravenous or subcutaneous injection into the subject. Injection of the viral vectors into a spinal fluid can also be used as a mode of administration, especially in the case of neuro-degenerative diseases. Following injection, the viral vectors circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can include, but are not limited to, viral vectors, viral genome, plasmids, phagemids and the like. Transfection vehicles such as liposomes can also be used to introduce the non-viral vectors described above into recipient cells within the inoculated area. Such transfection vehicles are known to one skilled within the art.

The following method resulted in the production of a stable RNA polymerase enzyme. The RNA polymerase enzyme has exhibited the properties disclosed above.

The colonies of 2a-JBWA-transformed E. coli (Tuner strain) were grown on Agar (+Amp) plates. Single colonies were transferred to 5 ml TB medium (−Amp) and grown to saturation at 37° C. (overnight), then 1 ml of the bacterial solution was used to inoculate 250 ml of pre-warmed TB medium, which was grown until absorption 0.8₆₀₀ was reached and then chilled on ice for 30 minutes. The 2a protein expression was induced in the chilled bacterial culture with 0.4 mM IPTG. The induced bacteria were re-shaken for five hours at room temperature and pelleted by centrifugation at 6000 rpm (1 minute). The bacterial pellet was quickly frozen with liquid nitrogen and stored for further use at −80° C.

To extract 2a protein, the bacterial pellet from 250 ml of IPTG-induced culture was re-suspended in 12 ml lysis cocktail (0.1M HEPES, pH 8.0; 0.1M KCl; 1.2 mM PMSF; 1 mM EDTA; 0.12 mM EGTA; 2 mM CaCl₂; 1.2 mg DNase I; 0.5 mg Lysozyme; 0.035% beta-mercaptoetanol; 10% glycerol; 0.1% IG-PAL).

The lysate was stirred for 30 minutes at 4° C. and ultracentrifuged for 1 hour at 30,000 rpm. The supernatant was transferred to Poly-Prep® Chromatography Column (Bio-Rad) pre-packed with 0.7 ml resuspended TALON resin that was allowed to sediment. The supernatant was filtered through the chromatography column by a gravity flow and discarded. The resin-bound fraction of the lysate was washed five times with 2 ml 50 mM Na₃PO₄, pH=7.0, 0.3M NaCl, 5 mM imidazole, each time including centrifugation for 10 seconds at low rpm, discarding the supernatant. The His-tagged 2a polymerase was eluted from the resin with 1 ml of a buffer containing 50 mM Na₃PO₄, pH=7.0, 0.3M NaCl and 0.17M imidazole. Four fractions of the eluant (250 μl each) were collected into separate tubes, dialyzed overnight in the storage buffer (50 mM Tris, pH 8.2; 50 mM KCl; 50 mM MgCl₂, 2 mM DTT; 0.15 mM PMSF; 0.75% Triton X-100; 50% glycerol), and stored at −20° C.

The in vitro 2a polymerase activity assays revealed that the enzyme expressed and purified by the described method is able to properly recognize the BMV-specific promoter for synthesis of (−) strand genomic (complement) RNA, and to initiate de novo RNA synthesis from this promoter, using a single nucleoside triphosphate (also referred to as one-nucleotide primer) that provides the 3′-hydroxyl for the addition of the next nucleotide. The optimal conditions for de novo initiation and RNA copying are: 3 μg RNA template; 5 U of 2a polymerase; 1 mM rATP, rGTP, rUTP, rCTP; 40 mM MgCl₂ , 0.6% Triton X-100; 8 mM DTT; 50 mM Tris-HCl, pH 8.0 and 3 U RNasin in a total volume of 30 μl. The reaction is incubated at 30° C. for 90 minutes.

The E. coli-expressed 2a polymerase is capable of in vitro copying any positive RNA template into negative or double-stranded RNA via primer-extension mechanism. This mode of initiation of RNA synthesis is promoter independent and it is primed by a short RNA oligonucleotide (60-200 nts) complementary to the template. The preferred conditions for primer-extension-driven initiation are: 3 μg RNA template; 6 μg of the oligonucleotide primer; 5 U 2a polymerase; 1 mM rATP, rGTP, rUTP, rCTP; 160 mM MgCl₂; 0.6% Triton X-100; 8 mM DTT; 50 mM Tris-HCl, pH 8.0 and 3 U RNasin in a total volume of 30 μl. The methodology can be used for large-scale production of an RNA polymerase of an RNA virus. Although the methodologies utilized here are commonly known within the biochemistry of protein expression in E. coli cells, one modification makes the extraction protocol much faster. That is, that the Tallon resin is added directly to the bacterial lysate which also prevents extensive proteolytic degradation of the final product. The use of the Tallon resin within a flow-through column significantly increases (up to 100%) the amount of the extracted 2a protein.

IPIG induces the transcription of 2a-RNA polymerase mRNA and its optimal concentration is 0.4 mM, +/−0.1 mM; however, other concentrations can also be used at a temperature of 25° C., for 5 hours after induction with IPTG. The tolerable temperature range is between 17° C. and 25° C.

The conditions for 2a-RNA polymerase extraction and storage include the use of a lysis/extraction buffer: 0.1M HEPES, pH 8.0; 0.1M KCl; 1.2 mM PMSF; 1 mM EDTA; 0.12 mM EGTA; 2 mM CaCl₂; 1.2 mg DNase I; 0.5 mg Lysozyme; 0.035% beta-mercaptoetanol; 10% glycerol; 0.1% IG-PAL; and a storage buffer: 50 mM Tris, pH 8.2; 50 mM KCl; 50 mM MgCl₂, 2 mM DTT; 0.150M PMSF; 0.75% Triton X-100; 50% glycerol.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

### REFERENCES

U.S. Pat. Nos. 4,866,042, 5,464,764 and 5,487,992
Dohi K, Mise K. Furusawa I, Okuno T 2002. RNA-dependent RNA polymerase complex of Brome mosaic virus: analysis of the molecular structure with monoclonal antibodies. J Gen Virol. 83:2879-90.
Figlerowicz M, Nagy P D, Tang N. Kao C C Buiarski J J 1998. Mutations in the N terminus of the brome mosaic virus polymerase affect genetic RNA-F1NA recombination. J. Virol. 72:9192-200.
Dzianoft A, Rauffer-Bruyere N, Bujarski J J. 2001. Studies on functional interaction between brome mosaic virus replicas proteins during RNA recombination, using combined mutants in vivo and in vitro. Virology 289:13749.
Wang Q M, Johnson R B, Chen D, Leveque V J, Ren J, Hockman M A, Abe K, Hachisu T, Kondo Y, Isaka Y, Sato A, Fujiwara T. 2004. Expression and purification of untagged full-length HCV NS5B RNA-dependent RNA polymerase. Protein Expr Punt. 35:304-12.
Rice, III., Charles Moen, Kolykhalov; Alexander A. May 21, 2002. Functional DNA clone for hepatitis C virus (HCV) and uses thereof. U.S. Pat. No. 6,392,028.
Hagedorn; Curt H. Oct. 8, 2002. Recombinant hepatitis C virus RNA replicase. U.S. Pat. No. 6,461,845.
Sambrook et al., Molecular Cloning: A Laboratory Manual, Cold Springs Harbor Laboratory, New York (1989, 1992).
Ausubel et al., Current Protocols in Molecular Biology, John Wiley and Sons, Baltimore, Md. (1989).
Chang et al., Somatic Gene Therapy, CRC Press, Ann Arbor, Mich. (1995).
Vega et at., Gene Targeting, CRC Press, Ann Arbor, Mich. (1995).
Butterworths, Vectors: A Survey of Molecular Cloning Vectors and Their Uses, Boston Mass. (1988).
Gilboa et al (1986).

What is claimed is:

1. A method of forming a stable RNA polymerase enzyme by:
expressing protein 2a in cells;
pelleting the cells expressing protein 2a;
macerating the cells expressing protein 2a to obtain a cell lysate; and
filtering the cell lysate through an affinity resin.

2. The method according to claim 1, further including dialyzing the lysate against a storage buffer.

3. The method according to claim 1, wherein said expressing step includes expressing protein 2a using a chemical compound.

4. The method according to claim 1, wherein said expressing step includes expressing protein 2a using IPTG.

5. The method according to claim 1, wherein said filtering step includes filtering through a His-tagged column.

6. A method of specific gene silencing by expressing protein 2a together with a specific RNA primer, thereby inducing specific gene silencing by copying only the selected RNA primer sequence.

7. A stable RNA polymerase enzyme for copying RNA in vitro.

8. The RNA polymerase enzyme according to claim 7, wherein said RNA polymerase enzyme forms double-stranded RNA.

9. The RNA polymerase enzyme according to claim 7, wherein said RNA polymerase enzyme selectively primes the RNA.

10. The RNA polymerase enzyme according to claim 7, wherein said RNA polymerase enzyme silences a gene during the copying.

11. The RNA polymerase enzyme according to claim 7, wherein said RNA polymerase enzyme is an active 2a RNA polymerase.

12. The RNA polymerase enzyme according to claim 7 for use in RNAi technology.
